(19) United States
(12) Patent Application Publication
(10) Pub. No.: US 2026/0207333 A1
(43) Pub. Date: Jul. 23, 2026

(54) REPLACEMENT HEART VALVE DELIVERY SYSTEM

(71) Applicant: BOSTON SCIENTIFIC SCIMED, INC., MAPLE GROVE, MN (US)

(72) Inventors: Levi Joel Wolterstorff, Saint Paul, MN (US); James M. Anderson, Corcoran, MN (US)

(73) Assignee: BOSTON SCIENTIFIC SCIMED, INC., MAPLE GROVE, MN (US)

(21) Appl. No.: 19/457,757

(22) Filed: Jan. 23, 2026

Related U.S. Application Data
(60) Provisional application No. 63/748,702, filed on Jan. 23, 2025.

(51) Int. Cl. A61F 2/24

(52) U.S. Cl. CPC A61F 2/2436; A61F 2/2418

(57) ABSTRACT

A replacement heart valve system includes an expandable framework with valve leaflets and a delivery system. The delivery system contains a handle assembly and an elongate shaft. The elongate shaft includes concentric outer, middle and inner shafts, with the heart valve implant secured to the middle shaft. The handle assembly features a laterally offset rotatable wheel between two control knobs. The wheel controls implant rotation via the middle shaft, while the knobs independently control translation of proximal and distal sheaths that constrain the implant. The sheaths can be retracted separately to allow controlled expansion of the implant's inflow and outflow regions. The handle assembly includes hubs coupled to each shaft and a rotation lock mechanism. When actuated, the control elements enable precise positioning and deployment of the replacement heart valve at the target site.

### CROSS REFERENCE TO RELATED APPLICATIONS

This application claims the benefit of priority of U.S. Provisional Application No. 63/748,702 filed January 23, 2025, the entire disclosure of which is hereby incorporated by reference.

### TECHNICAL FIELD

The disclosure relates generally to medical devices and more particularly to medical devices that are adapted for implanting stents and medical devices including a stent component.

### BACKGROUND

A wide variety of intracorporeal medical devices have been developed for medical use including, artificial heart valves for repair or replacement of diseased heart valves. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure relates generally to medical devices and more particularly to medical devices that are adapted for implanting stents and medical devices including a stent component. An example may be found in a replacement heart valve system. The replacement heart valve system includes a replacement heart valve implant including an expandable framework that is configured to shift from a radially collapsed configuration to a radially expanded configuration, a handle assembly having a longitudinal axis, and an elongate shaft extending distally from the handle assembly. The elongate shaft includes an outer shaft, a middle shaft that is rotatably disposed within the outer shaft, with the replacement heart valve implant releasably secured relative to the middle shaft, and an inner shaft that is disposed within the middle shaft. The handle assembly includes a rotatable wheel that has an axis of rotation that is laterally offset from and parallel to the longitudinal axis of the handle assembly. Rotation of the rotatable wheel causes a corresponding rotation of the middle shaft.

Alternatively or additionally, the middle shaft may rotate relative to the inner shaft and the outer shaft.

Alternatively or additionally, the handle assembly may further include a first rotatable knob that is operably coupled to the outer shaft such that rotation of the first rotatable knob causes translation of the outer shaft, and a second rotatable knob that is operably coupled to the inner shaft such that rotation of the second rotatable knob causes translation of the inner shaft.

Alternatively or additionally, the rotatable wheel may be disposed between the first rotatable knob and the second rotatable knob.

Alternatively or additionally, the elongate shaft may further include a proximal sheath that is operably coupled with the outer shaft such that rotation of the first knob causes translation of the proximal sheath, and a distal sheath that is operably coupled with the inner shaft such that rotation of the second knob causes translation of the distal sheath.

Alternatively or additionally, the proximal sheath and the distal sheath may constrain the expandable framework in the radially collapsed configuration when the proximal sheath and the distal sheath overlay the replacement heart valve implant.

Alternatively or additionally, translating the proximal sheath and the distal sheath away from the replacement heart valve implant may allow the expandable framework to expand into the radially expanded configuration.

Alternatively or additionally, the expandable framework may include a proximal outflow region and a distal inflow region.

Alternatively or additionally, the proximal sheath may be translated proximally to allow expansion of the proximal outflow region of the expandable framework without allowing expansion of the distal inflow region.

Alternatively or additionally, the distal sheath may be translated distally to allow expansion of the distal inflow region of the expandable framework without allowing expansion of the proximal outflow region.

Alternatively or additionally, the handle assembly may further include a middle shaft hub that is coupled to the middle shaft. The rotatable wheel may engage the middle shaft hub such that rotating the rotatable wheel in a first direction causes the middle shaft hub to rotate in an opposing second direction.

Another example may be found in an implant delivery system for delivering a replacement heart valve implant to a native heart valve. The implant delivery system includes an elongate shaft assembly having a middle shaft that is adapted to releasably secure the replacement heart valve implant thereto, a proximal sheath that is adapted to translate relative to the middle shaft and to reversibly cover a proximal portion of the implant, and a distal sheath that is adapted to translate relative to the middle shaft and to reversibly cover a distal portion of the implant. The implant delivery system includes a handle assembly that is secured relative to the elongate shaft assembly. The handle assembly includes a first rotatable knob that is adapted to cause the proximal sheath to translate when the first rotatable knob is rotated, a second rotatable knob that is adapted to cause the distal sheath to translate when the second rotatable knob is rotated, and a rotatable wheel that is disposed between the first rotatable knob and the second rotatable knob. The rotatable wheel is adapted to cause rotation of the middle shaft when the rotatable wheel is rotated, thereby altering a rotational position of the implant.

Alternatively or additionally, the handle assembly may further include a middle shaft hub that is adapted to rotate in response to the rotatable wheel being rotated.

Alternatively or additionally, the middle shaft may be operably coupled to the middle shaft hub such that the middle shaft rotates when the middle shaft hub rotates.

Alternatively or additionally, rotating the rotatable wheel in an arbitrary direction may cause the middle shaft hub to rotate in an opposing direction.

Alternatively or additionally, the handle assembly may further include an outer shaft hub that is adapted to translate in response to the first rotatable knob being rotated and an inner shaft hub that is adapted to translate in response to the second rotatable knob being rotated.

Alternatively or additionally, the elongate shaft assembly may further include an outer shaft that extends between the outer shaft hub and the proximal sheath.

Alternatively or additionally, the elongate shaft assembly may further include an inner shaft that extends within the outer shaft and extends between the inner shaft hub and the distal sheath.

Another example may be found in a handle assembly for delivering a replacement heart valve implant to a native heart valve. The handle assembly includes a first knob assembly and a second knob assembly. The first knob assembly includes a first rotatable knob, an axial adjustment knob that is disposed adjacent the first rotatable knob, and a first knob coupler. The second knob assembly includes a second rotatable knob, an axial screw, a rotation lock that is threadedly engaged with the axial screw, and a second knob coupler. The second knob assembly is free to rotate when the rotation lock is loose. Rotation of the axial adjustment knob permits axial translation of the second knob assembly. An implant delivery system includes the handle assembly and an elongate shaft assembly that extends distally of the handle assembly. The elongate shaft assembly includes an outer shaft that terminates within the first rotatable knob, a middle shaft that is rotatably disposed within the second knob coupler, and an inner shaft that extends through the second knob assembly.

This summary is an overview of some of the teachings of the present application and is not intended to be an exclusive or exhaustive treatment of the present subject matter. Further details are found in the detailed description and appended claims. Other features will be apparent to persons skilled in the art upon reading and understanding the following detailed description and viewing the drawings that form a part thereof, each of which is not to be taken in a limiting sense.

### BRIEF DESCRIPTION OF THE DRAWINGS

The disclosure may be more completely understood in consideration of the following detailed description in connection with the accompanying drawings, in which:

FIG. 1 is a perspective view of an illustrative replacement heart valve implant;

FIG. 2 is a schematic view of an illustrative heart valve delivery system for delivering the illustrative replacement heart valve implant of FIG. 1, with the replacement heart valve implant shown in a compressed configuration;

FIG. 3 is a schematic view of an illustrative heart valve delivery system for delivering the illustrative replacement heart valve implant of FIG. 1, with the replacement heart valve implant shown in an expanded configuration;

FIG. 4 is a cross-sectional view taken along the line 4-4 of FIG. 2;

FIG. 5 is a side view of a portion of an illustrative handle assembly forming part of the illustrative heart valve delivery system of FIGS. 2 and 3;

FIG. 6 is a perspective view of a portion of an illustrative handle assembly forming part of the illustrative heart valve delivery system of FIGS. 2 and 3;

FIG. 7 is a perspective view of an illustrative handle assembly that may be used as part of the illustrative heart valve delivery system of FIGS. 2 and 3; and

FIG. 8 is a cross-sectional view taken along the line 8-8 of FIG. 7.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the disclosure. Those skilled in the art will recognize that the various elements described and/or shown may be arranged in various combinations and configurations without departing from the scope of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including

“and/or” unless the content clearly dictates otherwise. It is to be noted that to facilitate understanding, certain features of the disclosure may be described in the singular, even though those features may be plural or recurring within the disclosed example(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some cases, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

In some instances, a replacement heart valve system includes a replacement heart valve implant having an expandable framework that is configured to shift from a radially collapsed configuration to a radially expanded configuration. In some cases, a plurality of valve leaflets may be disposed within and secured to the expandable framework. The replacement heart valve system includes a handle assembly having a longitudinal axis and an elongate shaft that extends distally from the handle assembly. The elongate shaft assembly includes an outer shaft, a middle shaft that is rotatably disposed within the outer shaft, and an inner shaft that is disposed within the middle shaft. The replacement heart valve implant is releasably secured relative to the middle shaft. The handle assembly includes a rotatable wheel that has an axis of rotation that is laterally offset from and parallel to the longitudinal axis of the handle assembly. Rotation of the rotatable wheel causes a corresponding rotation of the middle shaft.

In some cases, the middle shaft may rotate relative to the inner shaft and the outer shaft. In some cases, the handle assembly further includes a first rotatable knob that is operably coupled to the outer shaft such that rotation of the first rotatable knob causes translation of the outer shaft and a second rotatable knob that is operably coupled to the inner shaft such that rotation of the second rotatable knob causes translation of the inner shaft. In some cases, the rotatable wheel may be disposed between the first rotatable knob and the second rotatable knob. In some cases, the

elongate shaft may further include a proximal sheath that is operably coupled with the outer shaft such that rotation of the first knob causes translation of the proximal sheath and a distal sheath that is operably coupled with the inner shaft such that rotation of the second knob causes translation of the distal sheath. In some cases, the proximal sheath and the distal sheath may constrain the expandable framework in the radially collapsed configuration when the proximal sheath and the distal sheath overlay the replacement heart valve implant. Translating the proximal sheath and the distal sheath away from the replacement heart valve implant allows the expandable framework to expand into the radially expanded configuration, for example.

In some cases, the expandable framework may include a proximal outflow region and a distal inflow region. In some cases, the proximal sheath may be translated proximally to allow expansion of the proximal outflow region of the expandable framework without allowing expansion of the distal inflow region. In some cases, the distal sheath may be translated distally to allow expansion of the distal inflow region of the expandable framework without allowing expansion of the proximal outflow region. In some cases, the handle assembly may further include a middle shaft hub that is coupled to the middle shaft. The rotatable wheel may engage the middle shaft hub such that rotating the rotatable wheel in a first direction causes the middle shaft hub to rotate in an opposing second direction.

In some instances, an implant delivery system is for delivering a replacement heart valve implant to a native heart valve. The implant delivery system includes an elongate shaft assembly and a handle assembly that is secured relative to the elongate shaft assembly. The elongate shaft assembly includes a middle shaft that is adapted to releasably secure the replacement heart valve implant thereto, a proximal sheath that is adapted to translate relative to the middle shaft and to reversibly cover a proximal portion of the implant, and a distal sheath that is adapted to translate relative to the middle shaft and to reversibly cover a distal portion of the implant. The handle assembly includes a first rotatable knob that is adapted to cause the proximal sheath to translate when the first rotatable knob is rotated, a second rotatable knob that is adapted to cause the distal sheath to translate when the second rotatable knob is rotated, and a rotatable wheel that is disposed between the first rotatable knob and the second rotatable knob. The rotatable wheel is adapted to cause rotation of the middle shaft when the rotatable wheel is rotated, thereby altering a rotational position of the implant.

In some cases, the handle assembly may further include a middle shaft hub that is adapted to rotate in response to the rotatable wheel being rotated. The middle shaft may be operably coupled to the middle shaft hub such that the middle shaft rotates when the middle shaft hub rotates. In some cases, rotating the rotatable wheel in an arbitrary direction may cause the middle shaft hub to rotate in an opposing direction. In some cases, the handle assembly may further include an outer shaft hub that is adapted to translate in response to the first rotatable knob being rotated and an inner shaft hub that is adapted to translate in response to the second rotatable knob being rotated. In some cases, the elongate shaft assembly may further include an outer shaft extending between the outer shaft hub and the proximal sheath. In some cases, the elongate shaft assembly may further include an inner shaft that extends within the outer shaft and extends between the inner shaft hub and the distal sheath.

In some instances, a handle assembly may be used for delivering a replacement heart valve implant to a native heart valve. The handle assembly includes a first knob assembly and a second knob assembly. The first knob assembly includes a first rotatable knob, an axial adjustment knob that is disposed adjacent the first rotatable knob, and a first knob coupler. The second knob assembly includes a second rotatable knob, an axial screw, a rotation lock that is threadedly engaged with the axial screw, and a second knob coupler. The second knob assembly is free to rotate when the rotation lock is loose. Rotation of the axial adjustment knob permits axial translation of the second knob assembly. In some cases, an implant delivery system includes the aforementioned handle assembly and an elongate shaft assembly that extends distally of the handle assembly. The elongate shaft assembly may include an outer shaft terminating within the first rotatable knob, a middle shaft that is rotatably disposed within the second knob coupler, and an inner shaft that extends through the second knob assembly.

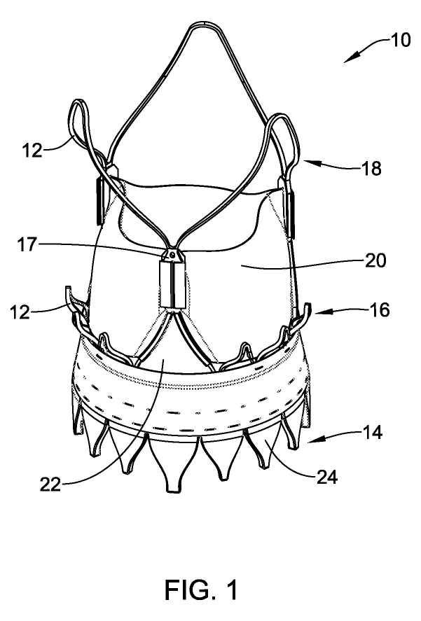

FIG. 1 is a schematic view of a replacement heart valve implant 10. It should be appreciated that the replacement heart valve implant 10 can be any type of replacement heart valve (e.g., a mitral valve, an aortic valve, etc.). In use, the replacement heart valve implant 10 may be implanted (e.g., surgically or through transcatheter delivery) in a mammalian heart. The replacement heart valve implant 10 can be configured to allow one-way flow through the replacement heart valve implant 10 from an inflow end to an outflow end.

For the purpose of this disclosure, the discussion herein is directed toward use in treating a native heart valve such as the aortic valve and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

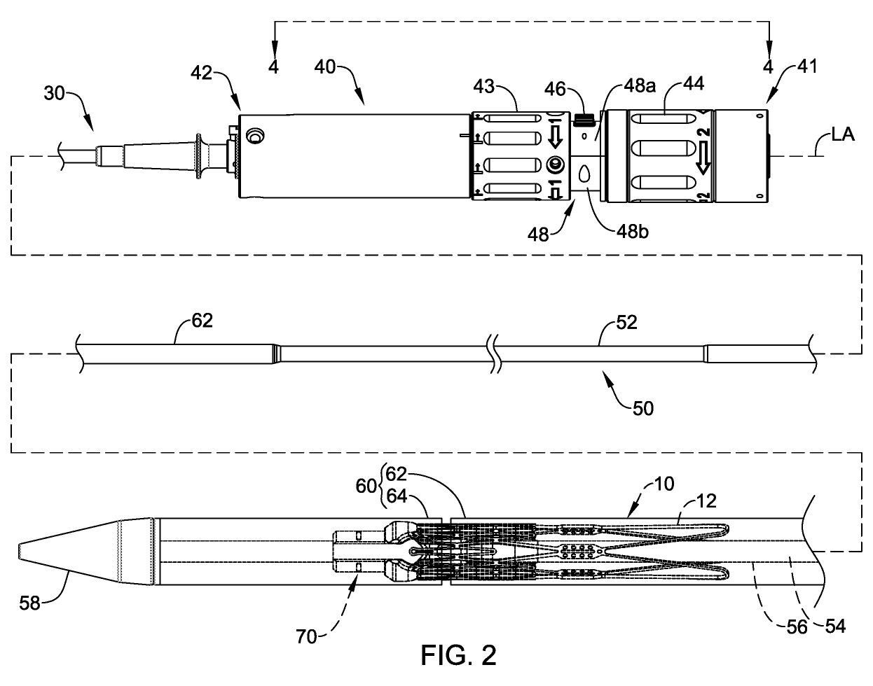

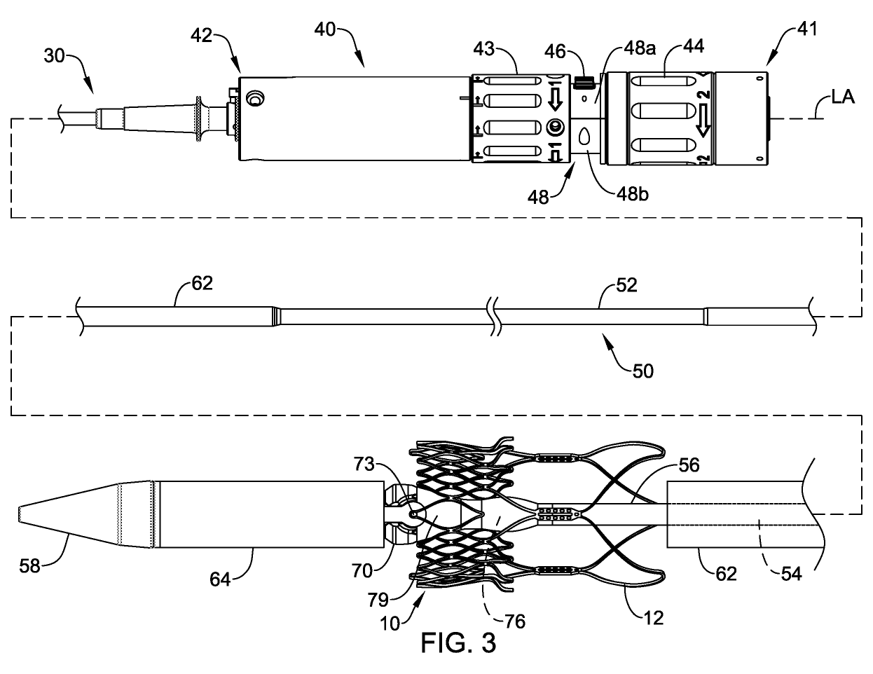

The replacement heart valve implant 10 may include an expandable framework 12 defining a central lumen. In some cases, the expandable framework 12 may have a substantially circular cross-section. In some cases, the expandable framework 12 can have a non-circular (e.g., D-shaped, elliptical, etc.) cross-section. Some suitable but non-limiting examples of materials that may be used to form the expandable framework 12, including but not limited to metals and metal alloys, composites, ceramics, polymers, and the like, are described below. The expandable framework 12 and/or the replacement heart valve implant 10 may be configured to shift between a radially collapsed configuration (e.g., FIG. 2) and a radially expanded configuration (e.g., FIGS. 1 and 3). In some cases, the expandable framework 12 may be self-expanding from the radially collapsed configuration to the radially expanded configuration. In some cases, the expandable framework 12 may be self-biased toward the radially expanded configuration. In some cases, the expandable framework 12 may be mechanically expandable from the radially collapsed configuration to the radially expanded configuration. In some cases, the expandable framework 12 may be balloon expandable from the radially collapsed configuration to the radially expanded configuration. Other configurations are also contemplated. In some cases, the expandable framework 12 may include and/or define a plurality of interstices (e.g., openings) through the expandable framework 12.

In some cases, the expandable framework 12 may include and/or define a lower crown 14 proximate an inflow end, an upper crown 16 proximate an outflow end, and a plurality of stabilization arches 18 extending downstream from the outflow end. In some cases, the lower crown 14 may be disposed at the inflow end. In some cases, the upper crown 16 may be disposed at the outflow end. In some cases, the expandable framework 12 may include a tubular wall defining the central lumen, the inflow end, the outflow end, the lower crown 14, and/or the upper crown 16.

In some cases, the expandable framework 12 may include and/or define a plurality of commissure posts 17 proximate the outflow end. In some cases, the plurality of commissure posts 17 may at least partially define the outflow end. Other configurations are also contemplated. In some cases, the plurality of commissure posts 17 may be disposed longitudinally and/or axially between the upper crown 16 and the plurality of stabilization arches 18. In some cases, the plurality of stabilization arches 18 may extend downstream of and/or away from the upper crown 16 and/or the plurality of commissure posts 17 in a direction opposite the lower crown 14. In some cases, the upper crown 16 may be disposed longitudinally and/or axially between the lower crown 14 and the plurality of stabilization arches 18. In some cases, the upper crown 16 may be disposed longitudinally and/or axially between the lower crown 14 and the plurality of commissure posts 17.

In some cases, the replacement heart valve implant 10 may include a proximal portion and a distal portion. In some cases, orientation of the replacement heart valve implant 10 may be related to an implant delivery device and/or a direction of implantation relative to a treatment site (e.g., a native heart valve, the aortic valve, etc.). In some cases, the proximal portion may include the outflow end and/or the plurality of stabilization arches 18. In some cases, the proximal portion may include the plurality of commissure posts 17, the upper crown 16, and/or the plurality of valve leaflets 20. In some cases, the distal portion may include the inflow end and/or the lower crown 14. Other configurations are also contemplated.

In some cases, the replacement heart valve implant 10 may include a plurality of valve leaflets 20 disposed within the central lumen. The plurality of valve leaflets 20 may be coupled, secured, and/or fixedly attached to the expandable framework 12. In at least some cases, the plurality of valve leaflets 20 may be coupled, secured, and/or fixedly attached to the expandable framework 12 at the plurality of commissure posts 17 to form and/or define a plurality of commissures. Each of the plurality of valve leaflets 20 may include a root edge coupled to the expandable framework 12 and a free edge (e.g., a coaptation edge) movable relative to the root edge to coapt with the free edges of the other valve leaflets along a coaptation region. In some cases, the plurality of valve leaflets 20 can be integrally formed with each other, such that the plurality of valve leaflets 20 is formed as a single unitary and/or monolithic unit. In some cases, the plurality of valve leaflets 20 may be formed integrally with other structures such as an inner skirt 22 and/or an outer skirt 24, base structures, liners, or the like.

The plurality of valve leaflets 20 may be configured to substantially restrict fluid from flowing through the replacement heart valve implant 10 in a closed position. For example, in some cases, the free edges of the plurality of valve leaflets 20 may move into coaptation with one another in the closed position to substantially restrict fluid from flowing through the replacement heart valve implant 10. The free edges of the plurality of valve leaflets 20 may be moved apart from each other in an open position to permit fluid flow through the replacement heart valve implant 10. In FIG. 1, the plurality of valve leaflets 20 is shown in the open position or in a partially open position (e.g., a neutral position) that the plurality of valve leaflets 20 may move to when unbiased by fluid flow.

In some cases, the plurality of valve leaflets 20 may include a polymer, such as a thermoplastic polymer. In some cases, the plurality of valve leaflets 20 may include at least 50 percent by weight of a polymer. In some cases, the plurality of valve leaflets 20 may be formed from bovine pericardium, porcine pericardium, or other living tissue. Other configurations and/or materials are also contemplated.

In some cases, the replacement heart valve implant 10 may include an inner skirt 22 disposed on and/or extending along an inner surface of the expandable framework 12. In at least some cases, the inner skirt 22 may be fixedly attached to the expandable framework 12. The inner skirt 22 may direct fluid, such as blood, flowing through the replacement heart valve implant 10 toward the plurality of valve leaflets 20. In at least some cases, the inner skirt 22 may be fixedly attached to and/or integrally formed with the plurality of valve leaflets 20. The inner skirt 22 may ensure the fluid flows through the central lumen of the replacement heart valve implant 10 and does not flow around the plurality of valve leaflets 20 when they are in the closed position.

In some cases, the replacement heart valve implant 10 can include an outer skirt 24 disposed on and/or extending along an outer surface of the expandable framework 12. In some cases, the outer skirt 24 may be disposed at and/or adjacent the lower crown 14. In some cases, the outer skirt 24 may be disposed between the expandable framework 12 and the vessel wall in order to prevent fluid, such as blood, flowing around the replacement heart valve implant 10 and/or the expandable framework 12 in a downstream direction. The outer skirt 24 may ensure the fluid flows through the replacement heart valve implant 10 and does not flow around the replacement heart valve implant 10, so as to ensure that the plurality of valve leaflets 20 can stop the flow of fluid when in the closed position.

In some cases, the inner skirt 22 may include a polymer, such as a thermoplastic polymer. In some cases, the inner skirt 22 may include at least 50 percent by weight of a polymer. In some cases, the outer skirt 24 may include a polymer, such as a thermoplastic polymer. In some cases, the outer skirt 24 may include at least 50 percent by weight of a polymer. In some cases, one or more of the plurality of valve leaflets 20, the inner skirt 22, and/or the outer skirt 24 may be formed of the same polymer or polymers. In some cases, the polymer may be a polyurethane. In some cases, the inner skirt 22 and/or the outer skirt 24 may be substantially impervious to fluid. In some cases, the inner skirt 22 and/or the outer skirt 24 may be formed from a thin tissue (e.g., bovine pericardium, porcine pericardium, etc.). In some cases, the inner skirt 22 and/or the outer skirt 24 may be formed from a coated fabric material. In some cases, the inner skirt 22 and/or the outer skirt 24 may be formed from a nonporous and/or impermeable fabric material. Other configurations are also contemplated. Some suitable but non-limiting examples of materials that may be used to form the inner skirt 22 and/or the outer skirt 24 including but not limited to polymers, composites, and the like, are described below.

FIG. 2 is a schematic view of a replacement heart valve system including the replacement heart valve implant 10 and an implant delivery system 30 for delivering a replacement heart valve implant to a native heart valve (e.g., the aortic valve). The implant delivery system 30 may be compatible with and/or usable with the replacement heart valve implant 10. In FIG. 2, some elements that would be hidden from view are shown in phantom to show relative positioning. In FIGS. 2 and 3, only the expandable framework 12 of the replacement heart valve implant 10 is shown. Other elements of the replacement heart valve implant 10 have been omitted to improve clarity. It should also be noted that FIGS. 2 and 3 include at least one change of scale (e.g., all parts of the figure are not drawn to the same scale) to improve viewability and show additional detail of selected aspects of the implant delivery system 30.

The implant delivery system 30 may include a handle assembly 40 and an elongate shaft assembly 50 extending distally from the handle assembly 40. The handle assembly 40 may include a first end 41 and a second end 42 opposite the first end 41. The elongate shaft assembly 50 may extend distally from the second end 42 of the handle assembly 40. The handle assembly 40 may include one or more rotatable knobs. In some cases, the one or more rotatable knobs may include a first rotatable knob 43 and a second rotatable knob 44. In at least some cases, the first rotatable knob 43 and/or the second rotatable knob 44 may be configured to rotate about a central longitudinal axis LA of the implant delivery system 30 and/or the handle assembly 40.

In some cases, a distal portion of the implant delivery system 30 and/or the elongate shaft assembly 50 may include an implant holding portion 60 configured to engage with and/or constrain the replacement heart valve implant 10 and/or the expandable framework 12 in the radially collapsed configuration, as seen in FIG. 2.

In some cases, the elongate shaft assembly 50 may include an inner shaft 54 axially secured to the handle assembly 40. In some cases, the elongate shaft assembly 50 may comprise an outer shaft 52 axially secured to the handle assembly 40 and disposed about the inner shaft 54. In the context of this disclosure, “axially secured” (and/or variants thereof) generally means that the feature or element is not free floating in an axial direction relative to another feature or element. For example, the feature or element may not be permitted to move freely in an axial direction or on its own but in some cases may be movable in an axial direction using a means or mechanism for controlled movement. In some cases, the inner shaft 54 may be slidably disposed within a lumen of the outer shaft 52.

In some cases, the inner shaft 54 may be rotationally decoupled from the handle assembly 40. In some cases, the outer shaft 52 may be rotationally decoupled from the handle assembly 40. In the context of this disclosure, “rotationally decoupled” (and/or variants thereof) generally means that the feature or element is permitted to rotate independently of and/or relative to another feature or element. For example, rotation of the handle assembly 40 may not be transferred to the inner shaft 54 and/or the outer shaft 52, or the inner shaft 54 and/or the outer shaft 52 does not rotate if/when the handle assembly 40 is rotated.

In some cases, the elongate shaft assembly 50 may include a distal tip 58 fixedly secured to a distal end of the inner shaft 54 distal of an implant holding portion 60. In some cases, the inner shaft 54 may extend distally from the handle assembly 40 within the outer shaft 52 to the distal tip 58 disposed distal of the implant holding portion 60. In some cases, the implant holding portion 60 may include a proximal sheath 62 and a distal sheath 64. In some cases, the proximal sheath 62 and/or the distal sheath 64 may be formed from a polymeric material. In some cases, the proximal sheath 62 and/or the distal sheath 64 may include a reinforcing structure disposed therein and/or thereon. In some cases, the reinforcing structure may be a coil, a mesh, one or more filaments, bands, or strips, or another suitable structure. Other configurations are also contemplated.

In some cases, the elongate shaft assembly 50 may include a middle shaft 56 disposed within and/or radially inward of the outer shaft 52 and about and/or radially outward of the inner shaft 54. In some cases, the middle shaft 56 may be fixedly attached to the handle assembly 40. In some cases, the inner shaft 54 may be slidably disposed within a lumen of the outer shaft 52 and/or the middle shaft 56. In at least some cases, the inner shaft 54 and the outer shaft 52 are each axially translatable relative to the middle shaft 56 independently of each other. For example, the inner shaft 54 may be translated relative to the middle shaft 56 without translating the outer shaft 52 relative to the middle shaft 56, and vice versa.

In some cases, the proximal sheath 62 may be axially secured to the outer shaft 52. In some cases, the distal tip 58 may be fixedly attached to the inner shaft 54. In some cases, the distal sheath 64 may be axially secured to the distal tip 58 and/or the inner shaft 54. In some cases, the distal sheath 64 may be axially secured to the inner shaft 54 via the distal tip 58 (e.g., the distal sheath 64 is axially secured to the distal tip 58 which is fixedly attached to the inner shaft 54). In some cases, the inner shaft 54 may include and/or at least partially define a guidewire lumen extending therethrough. In some cases, the guidewire lumen may extend through the handle assembly 40. In some cases, the distal sheath 64 may be rotationally decoupled from the distal tip 58 and/or the inner shaft 54. In some cases, the distal sheath 64 may be fixedly attached to the inner shaft 54 and/or the distal tip 58. In some cases, the distal sheath 64 may extend proximally from the distal tip 58. In some cases, the inner shaft 54 may include and/or at least partially define a guidewire lumen extending therethrough. In some cases, the guidewire lumen may extend through the handle assembly 40.

In some cases, the handle assembly 40 may be configured to manipulate and/or translate the proximal sheath 62 and/or the distal sheath 64 relative to each other using the first rotatable knob 43 and/or the second rotatable knob 44. In some cases, the handle assembly 40 may be configured to manipulate and/or translate the inner shaft 54 and/or the distal sheath 64 relative to the elongate shaft assembly 50, the outer shaft 52, the middle shaft 56, and/or the proximal sheath 62. In some cases, the handle assembly 40 may be configured to manipulate and/or translate the outer shaft 52 and/or the proximal sheath 62 relative to the elongate shaft assembly 50, the inner shaft 54, the middle shaft 56, and/or the distal sheath 64. In some cases, the handle assembly 40 may be configured to axially move the inner shaft 54 relative to the outer shaft 52 and/or the middle shaft 56. In some cases, the handle assembly 40 may be configured to axially move the outer shaft 52 relative to the inner shaft 54 and/or the middle shaft 56.

During delivery of the replacement heart valve implant 10 to a treatment site (e.g., the native heart valve, the aortic valve, etc.), the replacement heart valve implant 10 and/or the expandable framework 12 may be disposed at least partially within the proximal sheath 62 and/or the distal sheath 64 in the radially collapsed configuration when the implant holding portion 60 is disposed in a delivery configuration (e.g., FIG. 2). In some cases, the proximal sheath 62 and/or the distal sheath 64 may collectively define the implant holding portion 60 of the implant delivery system 30. In some cases, the implant holding portion 60 may be configured to constrain the replacement heart valve implant 10 and/or the expandable framework 12 in the radially collapsed configuration when the implant holding portion 60 is disposed in the delivery configuration (e.g., FIG. 2). In some cases, the replacement heart valve implant 10 and/or the expandable framework 12 may be releasably coupled to the inner shaft 54, the middle shaft 56, and/or a stent holder 70 (described in more detail below) when the replacement heart valve implant 10 and/or the expandable framework 12 is constrained within the implant holding portion 60 of the implant delivery system 30 in the radially collapsed configuration.

In some cases, the proximal sheath 62 may be configured to cover the proximal portion and/or the outflow end of the replacement heart valve implant 10 and/or the expandable framework 12 in the radially collapsed configuration when the implant holding portion 60 is disposed in the delivery configuration, and the distal sheath 64 may be configured to cover the distal portion and/or the inflow end of the replacement heart valve implant 10 and/or the expandable framework 12 in the radially collapsed configuration when the implant holding portion 60 is disposed in the delivery configuration. In some cases, the proximal sheath 62 may be disposed adjacent to the distal sheath 64 in the delivery configuration. In some cases, the proximal sheath 62 may abut or be axially spaced from the distal sheath 64 in the delivery configuration. In some cases, the proximal sheath 62 may be axially spaced apart from the distal sheath 64 in the delivery configuration by less than twenty percent, or less than fifteen percent, or less than ten percent, or less than five percent of an overall length of the replacement heart valve implant 10 and/or the expandable framework 12. Other configurations are also contemplated.

After advancing the replacement heart valve system and/or the implant delivery system 30 to a position adjacent the native heart valve (e.g., the aortic valve), the replacement heart valve implant 10 and/or the expandable framework 12 may be deployed within the native heart valve (e.g., the aortic valve). In some cases, rotation of the replacement heart valve implant 10 may be required to properly align the plurality of commissure posts 17 and/or the plurality of valve leaflets 20 with the corresponding aspects of the native heart valve (e.g., the aortic valve). In order to rotate the replacement heart valve implant 10 within the anatomy, the handle assembly 40 may be rotated about its central longitudinal axis. Due to the tortuous vasculature that the elongate shaft assembly 50 may be disposed in, friction and/or resistance to rotation may cause torque to build up within the elongate shaft assembly 50. If too much torque builds up, one or more portions of the elongate shaft assembly 50, such as the outer shaft 52 for example, may fracture or fail. As a result, it may be desirable to transmit torque along the elongate shaft assembly 50 without rotating and/or transmitting the torque to the outer shaft 52. Accordingly, in some cases, the outer shaft 52 may be rotatable relative to the handle assembly 40, the inner shaft 54, the middle shaft 56, and/or the replacement heart valve implant 10.

Similarly, friction between the replacement heart valve implant 10 and the implant holding portion 60, the proximal sheath 62, and/or the distal sheath 64 may impede rotation of the replacement heart valve implant 10 and/or may cause damage to the replacement heart valve implant 10 as the replacement heart valve implant 10 is rotated if the implant holding portion 60, the proximal sheath 62, and/or the distal sheath 64 do not also rotate. Accordingly, in some cases, the implant holding portion 60 may be rotatable relative to the outer shaft 52, the middle shaft 56, and/or the inner shaft 54. In some cases, the proximal sheath 62 may be rotatable relative to the outer shaft 52 and/or the middle shaft 56. In some cases, the distal sheath 64 may be rotatable relative to the distal tip 58 and/or the inner shaft 54. In some cases, the distal sheath 64 may be rotatable relative to the outer shaft 52 and/or the middle shaft 56.

In some cases, rotation of the handle assembly 40 may transmit torque and/or rotation to the replacement heart valve implant 10 via the middle shaft 56, thereby causing the replacement heart valve implant 10 to rotate within the implant holding portion 60 relative to the inner shaft 54 and/or the outer shaft 52. In some cases, rotating the handle assembly 40 of the implant delivery system 30 may cause the replacement heart valve implant 10 to rotate within the implant holding portion 60, thereby causing the implant holding portion 60 to rotate relative to the outer shaft 52.

As discussed herein, the middle shaft 56 may rotate within and/or relative to the outer shaft 52 because the outer shaft 52 may be rotatable relative to and/or rotationally decoupled from the handle assembly 40. In some cases, the middle shaft 56 may rotate about and/or relative to the inner shaft 54 because the inner shaft 54 may be rotatable relative to and/or rotationally decoupled from the handle assembly 40. In some cases, the outer shaft 52 may be rotatable independently of the handle assembly 40 and/or the inner shaft 54. In some cases, the inner shaft 54 may be rotatable independently of the handle assembly 40 and/or the outer shaft 52. Additionally, the implant holding portion 60 may be rotatable relative to the outer shaft 52, the middle shaft 56, and/or the inner shaft 54 such that the implant holding portion 60, the proximal sheath 62, and/or the distal sheath 64 may rotate along with the replacement heart valve implant 10 to prevent damage to the replacement heart valve implant 10. In some cases, the implant holding portion 60, the proximal sheath 62, and/or the distal sheath 64 may be rotatable independently of the handle assembly 40, the outer shaft 52, the middle shaft 56, and/or the inner shaft 54.

Deploying the replacement heart valve implant 10 and/or the expandable framework 12 may include shifting the proximal sheath 62 and the distal sheath 64 of the implant holding portion 60 from the delivery configuration (e.g., FIG. 2) to a release configuration (e.g., FIG. 3) to deploy the replacement heart valve implant 10. In some cases, shifting the proximal sheath 62 and the distal sheath 64 of the implant holding portion 60 from the delivery configuration to the release configuration may include shifting the proximal sheath 62 and the distal sheath 64 of the implant holding portion 60 axially apart from each other. In some cases, in the release configuration, a distal end of the proximal sheath 62 may be axially spaced apart from a proximal end of the distal sheath 64 by a greater distance than in the delivery configuration. In some cases, in the release configuration, the distal end of the proximal sheath 62 is axially spaced apart from the proximal end of the distal sheath 64 by at least an overall length of the replacement heart valve implant 10.

In some cases, the implant holding portion 60 and/or the elongate shaft assembly 50 may include a stent holder 70, seen in FIGS. 2 and 3. In at least some cases, the stent holder 70 may be fixedly attached to the elongate shaft assembly 50. In some cases, the stent holder 70 may be fixedly attached to the middle shaft 56 of the elongate shaft assembly 50. In some cases, the stent holder 70 may be integrally formed with the elongate shaft assembly 50 and/or the middle shaft 56. In some cases, the stent holder 70 may be configured to engage the expandable framework 12 in the radially collapsed configuration and/or when the replacement heart valve implant 10 is constrained within the implant holding portion 60 of the implant delivery system 30. In some cases, the stent holder 70 may include at least one projection 73 configured to engage the expandable framework 12 in the radially collapsed configuration. In some cases, the at least one projection 73 may be configured to engage the inflow end of the expandable framework 12 in the radially collapsed configuration. In some cases, the at least one projection 73 may extend into and/or through interstices of the expandable framework 12. In some cases, the expandable framework 12 may include at least one mounting loop configured to receive and/or engage with the at least one projection 73. Other configurations are also contemplated.

The implant delivery system 30 and/or the elongate shaft assembly 50 may include a primary visual indicator 76 (e.g., FIG. 3) disposed within the replacement heart valve implant 10 when the replacement heart valve implant 10 and/or the expandable framework 12 is constrained within the implant holding portion 60 in the radially collapsed configuration. The primary visual indicator 76 may be configured and/or adapted to be visible under fluoroscopy with an imaging device. Other imaging means suitable for use with transcatheter surgical procedures are also contemplated. The implant delivery system 30 and/or the primary visual indicator 76 may be configured to cooperate with the imaging device to position the replacement heart valve implant 10 at a desired insertion depth within the native heart valve (e.g., the aortic valve). In some cases, the primary visual indicator 76 may be fixedly attached to the elongate shaft assembly 50 and/or the middle shaft 56 by a shrink wrap or by an adhesive element. In some cases, the primary visual indicator 76 may be and/or may include a marker band. In some cases, the primary visual indicator 76 may be at least partially radiopaque. In some cases, the primary visual indicator 76 may be completely radiopaque. Other configurations are also contemplated.

As noted, the implant delivery system 30 may be rotated within the anatomy by rotating the handle assembly 40 about the longitudinal axis LA. In some cases, rotating the handle assembly 40 a certain distance about the longitudinal axis LA may not result in the distal portion of the implant delivery system 30, including the implant holding portion 60 and the replacement heart valve implant 10 held within the implant holding portion 60, rotating a corresponding amount as a result of frictional forces within the implant delivery system 30 as well as possible interactions with the anatomy. For example, a ten degree rotation of the handle assembly 40 may only result in a corresponding seven degree or eight degree rotation of the implant holding portion 60. Attempting to rotate the handle assembly 40 a few more degrees in order to achieve a ten degree rotation of the implant holding portion 60 may result in achieving the desired ten degree (in total) rotation of the implant holding portion 60. In some cases, attempting to rotate the handle assembly 40 a few more degrees in order to achieve the desired ten degree (in total) rotation may result in overshooting the desired rotation when the distal portion of the implant delivery system 30 overcomes a frictional impediment and advances more degrees than intended.

In some cases, the handle assembly 40 may include a rotatable wheel 46 that extends radially outwardly from a hub ring 48. In some cases, the hub ring 48 may be a unitary structure. In some cases, the hub ring 48 may be a two-part structure, including an upper hub ring 48a and a lower hub ring 48b that fit together to form the hub ring 48. The rotatable wheel 46 extends through the hub ring 48 and engages internal components of the handle assembly 40 such that rotation of the rotatable wheel 46 results in a corresponding rotation of particular components of the elongate shaft assembly 50, thereby rotating the replacement heart valve implant 10 held within the implant holding portion 60.

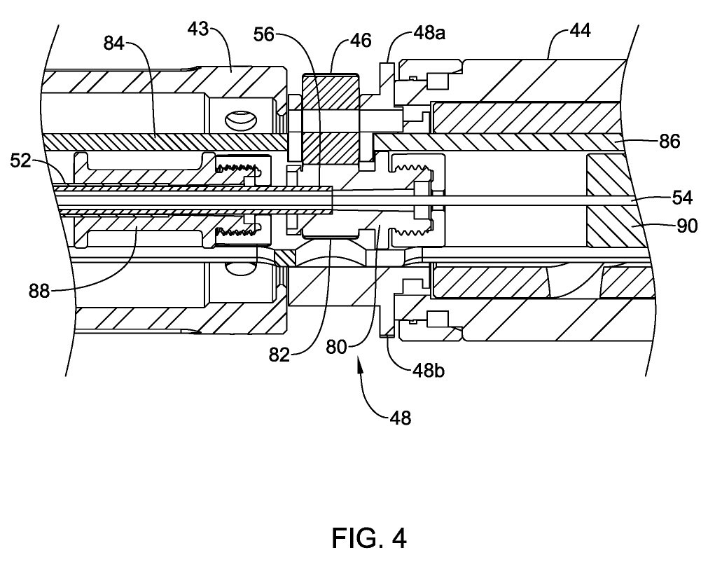

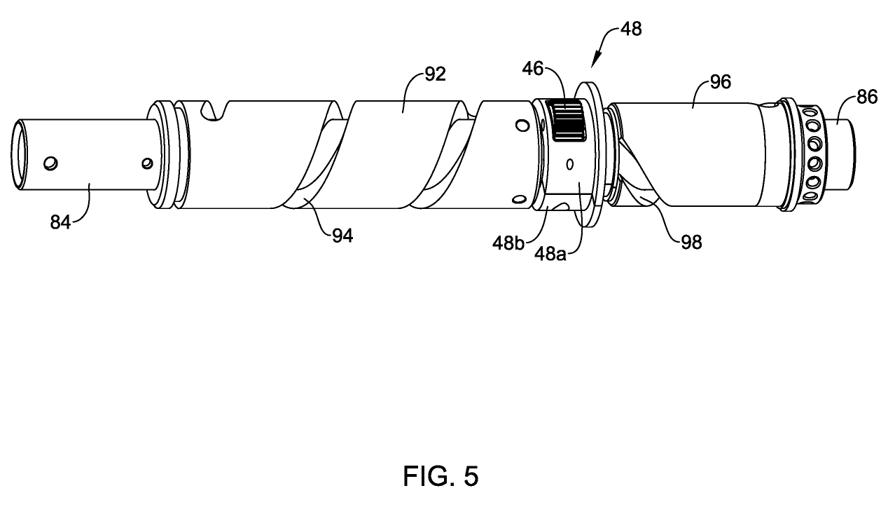

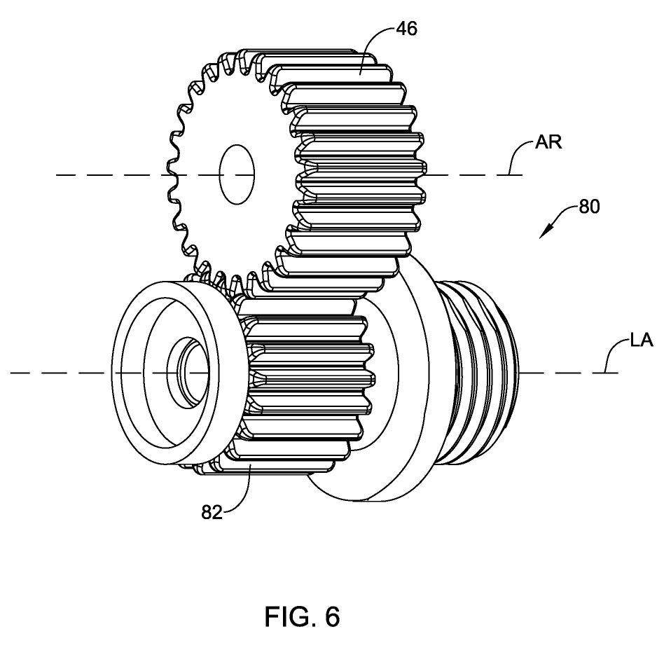

FIGS. 4, 5 and 6 provide details regarding some of the internal components of the handle assembly 40, including the internal components that allow the rotatable wheel 46 to provide small rotations to the replacement heart valve 10. FIG. 4 is a cross-sectional view taken along the line 4-4 of FIG. 2, FIG. 5 shows the handle assembly 40 with the first rotatable knob 43 and the second rotatable knob 44 removed to show structure underneath, and FIG. 6 is a perspective view of the rotatable wheel 46 and a middle shaft hub 80 that includes or carries a pinion gear 82. In some cases, the pinion gear 82 is integrally formed with the middle shaft hub 80. In some cases, the rotatable wheel 46 has an axis of rotation AR that is parallel with, but laterally offset from, the longitudinal axis LA.

The handle assembly 40 includes a first central tube 84 and a second central tube 86. An outer shaft hub 88 is disposed within the first central tube 84. In some cases, the outer shaft hub 88 is slidingly disposed within the first central tube 84. In some cases, the outer shaft 52 is operably coupled to the outer shaft hub 88 such that the outer shaft 52 translates when the outer shaft hub 88 translates. An inner shaft hub 90 is disposed within the second central tube 86. In some cases, the inner shaft hub 90 is slidingly disposed within the second central tube 86. In some cases, the inner shaft 54 is operably coupled to the inner shaft hub 90 such that the inner shaft 54 translates when the inner shaft hub 90 translates. The hub ring 48 is disposed between the first central tube 84 and the second central tube 86. In some cases, the middle shaft 56 is operably coupled to the middle shaft hub 80 such that the middle shaft 56 rotates when the middle shaft hub 80 rotates (in response to the rotatable wheel 46 engaging the pinion gear 82 of the middle shaft hub 80). In some cases, rotating the middle shaft 56 causes a corresponding rotation of the replacement heart valve 10. In some cases, the rotatable wheel 46 may be replaced by a lever that is able to engage the middle shaft hub 80. In some cases, the handle assembly 40 may include a lock or guard (not shown) that would discourage or even prevent accidental actuation of the rotatable wheel 46.

As illustrated in FIG. 5, the handle assembly 40 includes a first translation member 92 including a helical slot 94 and a second translation member 96 including a helical slot 98. The first translation member 92 overlies the first central tube 84 while the second translation member 96 overlies the second central tube 86. In some cases, the first translation member 92 is slidingly disposed over the first central tube 84 and the second translation member 96 is slidingly disposed over the second central tube 86. In some cases, the first rotatable knob 43 overlies the first translation member 92 and the second rotatable knob 44 overlies the second translation member 96.

In some cases, an element such as a rod, a threaded rod, or a bolt may extend between the first rotatable knob 43 and engage the helical slot 94 disposed within the first translation member 92. As a result, rotation of the first rotatable knob 43 causes translation of the first translation member 92. The first translation member 92 may be operably coupled to the outer shaft hub 88 by the same element (rod, threaded rod, or bolt), and thus the outer shaft hub 88 may translate relative to the first central tube 84 in response to rotation of the first rotatable knob 43. Translation of the outer shaft hub 88 causes translation of the outer shaft 52. In some cases, the proximal sheath 62 may be operably coupled to the outer shaft 52, and thus rotation of the first rotatable knob 43 can cause translation of the proximal sheath 62.

In some cases, an element such as a rod, a threaded rod, or a bolt may extend between the second rotatable knob 44 and engage the helical slot 98 disposed within the second translation member 96. As a result, rotation of the second rotatable knob 44 causes translation of the second translation member 94. The second translation member 94 may be operably coupled to the inner shaft hub 90 by the same element (rod, threaded rod, or bolt), and thus the inner shaft hub 90 may translate relative to the second central tube 86 in response to rotation of the second rotatable knob 44. Translation of the inner shaft hub 90 causes translation of the inner shaft 54. In some cases, the distal sheath 64 may be operably coupled to the inner shaft 54, and thus rotation of the second rotatable knob 44 can cause translation of the distal sheath 64. It will be appreciated, then, that the first rotatable knob 43 and the second rotatable knob 44 may be used to partially deploy part or all of the expandable framework 12. Rotation of the first rotatable knob 43 can cause translation of the proximal sheath 62, thereby enabling expansion of the proximal (outflow end) of the expandable framework 12. Rotation of the second rotatable knob 44 can cause translation of the distal sheath 64, thereby enabling expansion of the distal (inflow end) of the expandable framework 12.

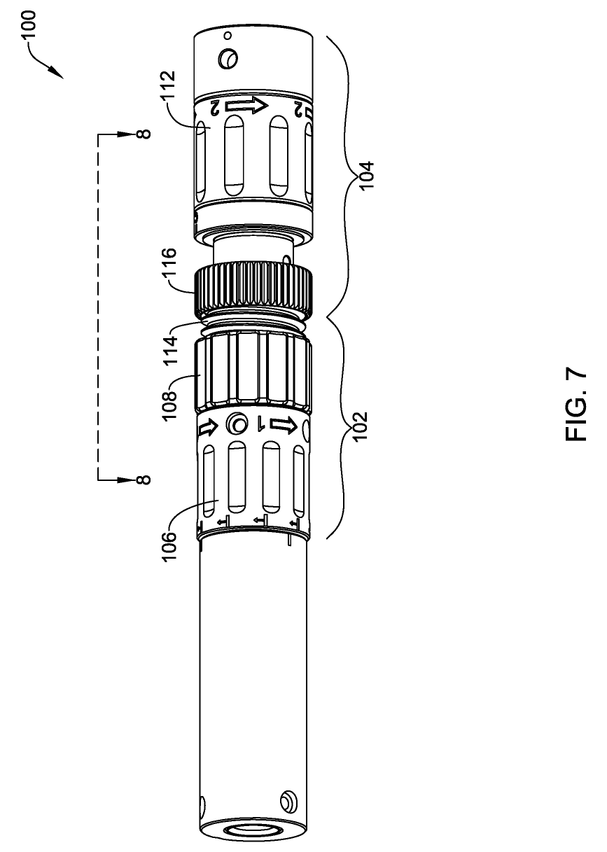

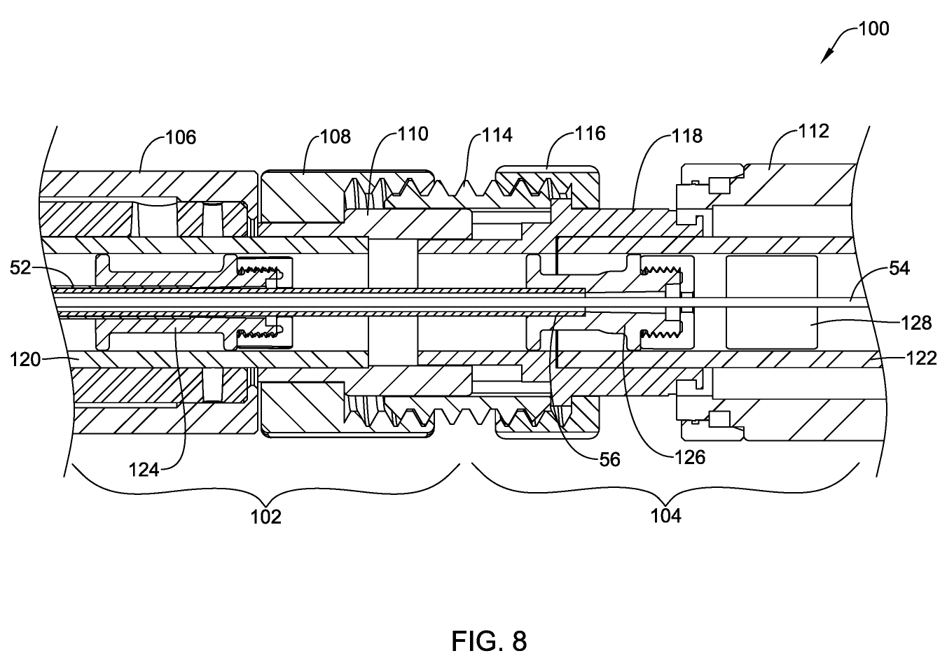

FIG. 7 is a perspective view of an illustrative handle assembly 100 that may be used in place of the handle assembly 40 shown as part of the heart valve delivery system 30 shown for example in FIGS. 2 and 3. FIG. 8 is a cross-sectional view of the handle assembly 100, taken along the line 8-8 of FIG. 7. In some cases, the handle assembly 100 provides the ability to make fine axial position adjustments. In some cases, the handle assembly 100 provides for free rotation in commissural alignment.

The handle assembly 100 includes a first knob assembly 102 and a second knob assembly 104. The first knob assembly 102 includes a first rotatable knob 106 that is essentially the same as the rotatable knob 43 shown as part of the handle assembly 40, an axial adjustment knob 108, and a first knob coupler 110. Rotating the first rotatable knob 106 will cause translation of the outer shaft 52 and hence will cause translation of the proximal sheath 62. The second knob assembly 104 includes a second rotatable knob 112 that is essentially the same as the rotatable knob 44 shown as part of the handle assembly 40, an axial screw 114, a rotation lock 116, and a second knob coupler 118 (FIG. 8). Rotating the second rotatable knob 112 will cause translation of the inner shaft 54 and hence will cause translation of the distal sheath 64.

In some cases, the handle assembly 100 includes a first central tube 120 and a second central tube 122. The handle assembly 100 includes an outer shaft hub 124 that is operably coupled with the outer shaft 52, a middle shaft hub 126 that is operably coupled with the middle shaft 56, and an inner shaft hub 128 that is operably coupled with the inner shaft 54. In some cases, the outer shaft hub 124 is disposed within the first central tube 120, the middle shaft hub 126 is disposed within the second central tube 122, and the inner shaft hub 128 is disposed within the second central tube 122. In some cases, the middle shaft hub 126 is fixedly secured within the second central tube 122, as is the second knob coupler 118.

In some cases, the second knob assembly 104 is free to rotate when the rotation lock 116 is loose. When the rotation lock 116 is tight, meaning that the rotation lock 116 is threadedly engaged with the axial screw 114, the second knob assembly 104 is prevented from rotating because the second knob coupler 118 is clamped against the axial screw 114 and is not able to rotate. In some cases, the axial screw 114 may include a keyway (not shown) or other structure that allow the axial screw 114 to translate, but prevents the axial screw 114 from rotating. In some cases, rotating the axial adjustment knob 108 creates translation of the second knob assembly 104. In some cases, friction may be used to limit or even prevent unwanted rotation of the axial adjustment knob 108.

The materials that can be used for the various components of the replacement heart valve system and the various elements thereof disclosed herein may include those commonly associated with medical devices. For simplicity purposes, the following discussion refers to the system. However, this is not intended to limit the devices, components, and methods described herein, as the discussion may be applied to other elements, members, components, or devices disclosed herein, such as, but not limited to, the replacement heart valve implant, the expandable framework, the plurality of valve leaflets, the implant delivery system, the handle assembly, the elongate shaft assembly, etc. and/or elements or components thereof.

In some cases, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some cases, the system and/or components thereof can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some cases, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some cases, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some cases, the system and/or other elements disclosed herein may include a fabric material disposed over or within the structure. The fabric material may be composed of a biocompatible material, such a polymeric material or biomaterial, adapted to promote tissue ingrowth. In some cases, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some cases, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni-Co-Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some cases, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A replacement heart valve system, comprising:
a replacement heart valve implant including an expandable framework configured to shift from a radially collapsed configuration to a radially expanded configuration;
a handle assembly having a longitudinal axis; and
an elongate shaft extending distally from the handle assembly, the elongate shaft including:
an outer shaft;
a middle shaft rotatably disposed within the outer shaft, wherein the replacement heart valve implant is releasably secured relative to the middle shaft; and
an inner shaft disposed within the middle shaft;
wherein the handle assembly includes a rotatable wheel that has an axis of rotation that is laterally offset from and parallel to the longitudinal axis of the handle assembly; and
wherein rotation of the rotatable wheel causes a corresponding rotation of the middle shaft.

2. The replacement heart valve system of claim 1, wherein the middle shaft rotates relative to the inner shaft and the outer shaft.

3. The replacement heart valve system of claim 1, wherein the handle assembly further comprises:
a first rotatable knob operably coupled to the outer shaft such that rotation of the first rotatable knob causes translation of the outer shaft; and
a second rotatable knob operably coupled to the inner shaft such that rotation of the second rotatable knob causes translation of the inner shaft.

4. The replacement heart valve system of claim 3, wherein the rotatable wheel is disposed between the first rotatable knob and the second rotatable knob.

5. The replacement heart valve system of claim 3, wherein the elongate shaft further comprises:
a proximal sheath operably coupled with the outer shaft such that rotation of the first knob causes translation of the proximal sheath; and
a distal sheath operably coupled with the inner shaft such that rotation of the second knob causes translation of the distal sheath.

6. The replacement heart valve system of claim 5, wherein the proximal sheath and the distal sheath constrain the expandable framework in the radially collapsed configuration when the proximal sheath and the distal sheath overlay the replacement heart valve implant.

7. The replacement heart valve system of claim 6, wherein translating the proximal sheath and the distal sheath away from the replacement heart valve implant allows the expandable framework to expand into the radially expanded configuration.

8. The replacement heart valve system of claim 5, wherein the expandable framework comprises a proximal outflow region and a distal inflow region.

9. The replacement heart valve system of claim 8, wherein the proximal sheath may be translated proximally to allow expansion of the proximal outflow region of the expandable framework without allowing expansion of the distal inflow region.

10. The replacement heart valve system claim 5, wherein the distal sheath may be translated distally to allow expansion of the distal inflow region of the expandable framework without allowing expansion of the proximal outflow region.

11. The replacement heart valve system of claim 1, wherein the handle assembly further comprises a middle shaft hub coupled to the middle shaft, and the rotatable wheel engages the middle shaft hub such that rotating the rotatable wheel in a first direction causes the middle shaft hub to rotate in an opposing second direction.

12. An implant delivery system for delivering a replacement heart valve implant to a native heart valve, the implant delivery system comprising:
an elongate shaft assembly including:
a middle shaft adapted to releasably secure the replacement heart valve implant thereto;
a proximal sheath adapted to translate relative to the middle shaft and to reversibly cover a proximal portion of the implant; and
a distal sheath adapted to translate relative to the middle shaft and to reversibly cover a distal portion of the implant; and
a handle assembly secured relative to the elongate shaft assembly, the handle assembly including:
a first rotatable knob that is adapted to cause the proximal sheath to translate when the first rotatable knob is rotated;
a second rotatable knob that is adapted to cause the distal sheath to translate when the second rotatable knob is rotated; and
a rotatable wheel disposed between the first rotatable knob and the second rotatable knob, the rotatable wheel adapted to cause rotation of the middle shaft when the rotatable wheel is rotated, thereby altering a rotational position of the implant.

13. The implant delivery system of claim 12, wherein the handle assembly further comprises a middle shaft hub that is adapted to rotate in response to the rotatable wheel being rotated.

14. The implant delivery system of claim 13, wherein the middle shaft is operably coupled to the middle shaft hub such that the middle shaft rotates when the middle shaft hub rotates.

15. The implant delivery system of claim 13, wherein rotating the rotatable wheel in an arbitrary direction causes the middle shaft hub to rotate in an opposing direction.

16. The implant delivery system of claim 12, wherein the handle assembly further comprises:
an outer shaft hub that is adapted to translate in response to the first rotatable knob being rotated; and
an inner shaft hub that is adapted to translate in response to the second rotatable knob being rotated.

17. The implant delivery system of claim 12, wherein the elongate shaft assembly further comprises an outer shaft extending between the outer shaft hub and the proximal sheath.

18. The implant delivery system of claim 12, wherein the elongate shaft assembly further comprises an inner shaft that extends within the outer shaft and extends between the inner shaft hub and the distal sheath.

19. A handle assembly for delivering a replacement heart valve implant to a native heart valve, the handle assembly comprising:
a first knob assembly including:
a first rotatable knob;
an axial adjustment knob disposed adjacent the first rotatable knob; and
a first knob coupler; and
a second knob assembly including:
a second rotatable knob;
an axial screw;
a rotation lock threadedly engaged with the axial screw; and
a second knob coupler;
wherein the second knob assembly is free to rotate when the rotation lock is loose; and
wherein rotation of the axial adjustment knob permits axial translation of the second knob assembly.

20. An implant delivery system, comprising:
the handle assembly of claim 19; and
an elongate shaft assembly extending distally of the handle assembly, the elongate shaft assembly comprising:
an outer shaft terminating within the first rotatable knob;
a middle shaft rotatably disposed within the second knob coupler; and
an inner shaft extending through the second knob assembly.
